(19) United States
(12) Patent Application Publication
(10) Pub. No.: US 2009/0036961 A1
(43) Pub. Date: Feb. 5, 2009

(54) MEDICAL USE ELECTRICAL LEAD INCLUDING A RADIO OPAGUE MARKER

(76) Inventors: Evan CHONG, South Strathfield (AU); Neil L. ANDERSON, Roseville (AU)

Correspondence Address: MORRISON & FOERSTER LLP, 755 PAGE MILL RD, PALO ALTO, CA, 94304-1018, US

(21) Appl. No.: 12/045,485

(22) Filed: Mar. 10, 2008

Related U.S. Application Data
(60) Provisional application No. 60/906,640, filed on Mar. 12, 2007.

(51) Int. Cl. A61N 1/05; A61B 5/0408

(52) U.S. Cl. 607/122; 604/103.1; 600/374

(57) ABSTRACT

A medical use electrical lead 10 includes an elongate body member 11 comprising an inner tubular member 12 defining a lumen 16 and a cover member 20 covering the inner tubular member 14. At least one electrode 22 is defined on an outer surface of the cover member 20. At least one pair of conductors 18 is arranged between the inner tubular member 14 and the cover member 20, the at least one pair of conductors 18 being in electrical communication with the at least one electrode 22 through the cover member 20. A radio opaque marker 28 circumscribes a periphery of the inner tubular member 14 and underlies the at least one electrode 22.

### CROSS-REFERENCE TO RELATED APPLICATIONS

This application claims priority from United States of America Provisional Patent Application No. 60/906,640, filed on Mar. 12, 2007, the disclosure of which is incorporated herein by reference in its entirety.

### FIELD

This invention relates, generally, to an electrical lead and, more particularly, to a medical use electrical lead which is desirably used as a component of a catheter.

### BACKGROUND

The applicant has devised a manufacturing technique for manufacturing a medical use electrical lead, particularly for use as an electrode sheath of a catheter, having an unimpeded lumen. This manufacturing technique embodies the use of a hollow, inner tubular member about which electrical conductors are wound. The electrical conductors are contained between the inner tubular member and an outer jacket on to which electrodes are applied.

A benefit of this manufacturing technique is that the helically wound electrical conductors provide greater flexibility to the electrical lead. In addition, by applying the electrodes on an outer surface of the jacket, greater flexibility of the electrodes themselves results.

Because the applied electrodes are relatively thin, the radio opacity of such electrodes may, at times, be inadequate. It would therefore be beneficial to provide improved radio opacity so that the positions of the electrodes can be more easily determined by a clinician using the medical use electrical lead.

### SUMMARY

According to a first aspect of the invention, there is provided a medical use electrical lead which includes
an elongate body member comprising an inner tubular member defining a lumen and a cover member covering the inner tubular member;
at least one electrode defined on an outer surface of the cover member;
at least one pair of conductors arranged between the inner tubular member and the cover member, the at least one pair of conductors being in electrical communication with the at least one electrode through the cover member; and
a radio opaque marker circumscribing a periphery of the inner tubular member and underlying the at least one electrode.

In this specification, the term “circumscribe” (and derivatives) is to be understood to mean, unless the context clearly indicates otherwise, that the radio opaque marker extends completely about the periphery of the inner tubular member.

Preferably, the radio opaque marker is formed by an elongate element arranged about the tubular member, the elongate element being configured to provide radio-opacity at at least the position of the at least one electrode on the body member. It will be appreciated that, in the case of a multi-electrode lead, each electrode has a radio opaque marker associated with it.

The elongate element may be a winding of a radio opaque material. The winding may be a metal wire which is biocompatible and which is sufficiently dense to be radio opaque under x-ray. For example, the wire may be of tantalum, platinum, tungsten, or the like.

Turns of the winding may have a smaller pitch in the region of the at least one electrode. The term “smaller pitch” means that the turns of the winding are closer together than the turns of the winding at regions of the body member free of electrodes.

At a distal region of the elongate body member, the winding may have a pitch greater than the pitch of the winding in the region of the at least one electrode to provide kink resistance at the distal region of the elongate body member.

The elongate element may be wound in an opposite sense to the at least one pair of conductors.

The elongate element may underlie the at least one pair of conductors. The elongate element may be covered by a sleeve interposed between the inner tubular member and the cover member. The elongate element may carry an insulating layer.

According to a second aspect of the invention, there is provided a method of forming a medical use electrical lead, the method including
providing an inner tubular member;
arranging at least one pair of conductors helically about the tubular member;
covering the at least one pair of conductors with a cover member;
forming at least one electrode at least partially about the cover member at a predetermined position and placing the electrode in electrical communication with the at least one pair of conductors; and
arranging a radio opaque marker about a periphery of the tubular member to circumscribe a periphery of the tubular member beneath the at least one electrode to provide radio-opacity at the location of the at least one electrode.

The method may include arranging the at least one radio opaque marker about the periphery of the tubular member prior to winding the at least one pair of conductors about the tubular member so that the at least one pair of conductors overlies the at least one radio opaque marker.

Preferably, the method includes forming the at least one radio opaque marker by winding an elongate element about the inner tubular member.

The method may include winding the elongate element in an opposite sense to the at least one pair of conductors.

Further, the method may include, in the region of the at least one electrode, winding the elongate element with a smaller pitch.

The method may include, at a distal region of the inner tubular member, winding the elongate element with a greater pitch than in the region of the at least one electrode to provide kink resistance to the distal region of the inner tubular member.

The method may include interposing a sleeve between the inner tubular member and the cover member and at least partially embedding the elongate element in the sleeve.

According to a third aspect of the invention, there is provided a catheter sheath which includes
an elongate body member comprising an inner tubular member defining a lumen and a cover member covering the inner tubular member;
at least one electrode defined on an outer surface of the cover member;
at least one pair of conductors arranged about the inner tubular member and covered by the cover member and in electrical communication with the at least one electrode through the cover member; and
a radio opaque marker circumscribing a periphery of the inner tubular member and underlying the at least one electrode.

The invention extends also to a catheter which includes a catheter sheath as described above. The catheter is, preferably, a cardiovascular catheter.

### BRIEF DESCRIPTION OF DRAWINGS

FIG. 1 shows a three dimensional view of a medical use electrical lead in accordance with an embodiment of the invention;

FIG. 2 shows a schematic, sectional side view of a part of the electrical lead; and

FIG. 3 shows a schematic side view of a catheter including the electrical lead functioning as a catheter sheath.

### DETAILED DESCRIPTION

In the drawings, reference numeral 10 generally designates a medical use electrical lead, in the form of a catheter sheath, or electrode sheath, for a catheter, in accordance with an embodiment of the invention.

The lead 10 comprises a body member 12 having an inner tubular member 14 defining a lumen 16. A plurality of electrical conductors 18 is helically wound about an outer surface of the inner tubular member 14. The electrical conductors 18 are covered by a cover member or jacket 20 of the body member 12. The tubular member 14 and the jacket 20 are of a thermoplastic elastomeric material and, preferably, are of polyester block amide such as that sold under the Registered Trademark, PEBAX®.

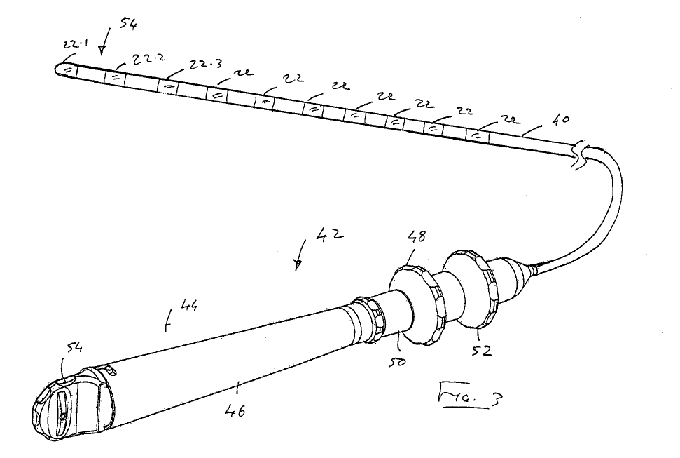

A plurality of electrodes 22 (FIG. 2) is applied about an outer surface 24 of the jacket 20. The electrodes 22 are arranged at longitudinally spaced intervals along the length of the jacket 20. The electrodes 22 are deposited in a suitable manner, for example, by pad printing or other deposition techniques to provide flexible electrodes.

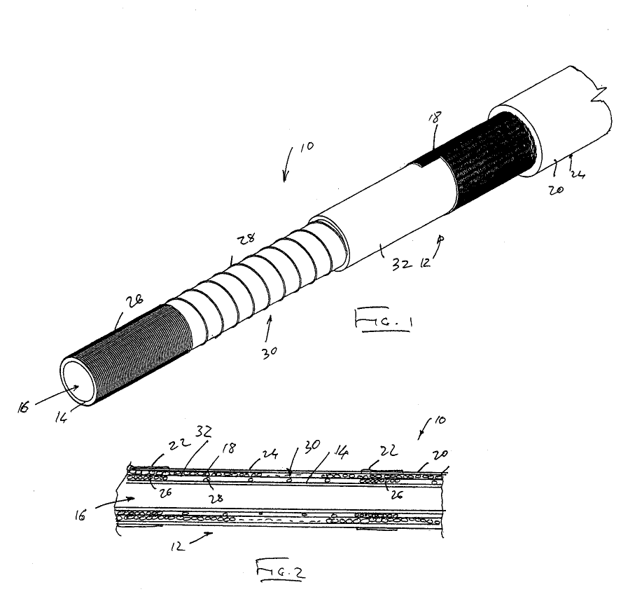

The electrical lead 10 includes a radio opaque marker 26 underlying each electrode 22 and circumscribing a periphery of the inner tubular member 14. More particularly, each radio opaque marker 26 is formed by a winding of an elongate element 28 of radio opaque material. More particularly, the winding 28 is wound helically about the inner tubular member 14. At the region of each electrode 22, the winding 28 is wound with a smaller pitch, i.e. with turns of the winding closer together, to form the radio opaque markers 26. The turns of the winding at each position of the radio opaque markers 26 can, if desired, be in abutting relationship whereas, between the electrodes 22, the turns of the winding 28 have a greater pitch as shown generally at 30 in FIGS. 1 and 2 of the drawings.

The winding 28 is arranged beneath the electrical conductors 18. In other words, the electrical conductors 18 are helically wound about the inner tubular member 14 over the winding 28. The winding 28 is embedded in, or underlies, a sleeve 32 of a plastics material.

To enable an electrical connection to be made between each electrode 22 and its associated conductors 18, a hole (not shown) is laser cut in the jacket 20 and is filled with a conductive adhesive. To inhibit separation of the conductors 18 when hole filling occurs the conductors 18 are parallel bonded. Each electrode 22 may have at least two parallel bonded conductors 18 (a copper-copper pair) associated with it. However, each electrode 22 may have four conductors 18 associated with it being a copper-copper pair for the supply of energy to the electrode 22 and a copper-constantin pair for a thermocouple. Parallel bonding of the conductors 18 inhibits exposing the underlying winding 28 to the conductive material and inhibits the formation of electrical cross-connections to the underlying winding 28. The provision of the sleeve 32 further reduces the likelihood of a short circuit developing between the conductors 18 and the underlying winding 28.

Preferably, the electrical conductors 18 and the winding 28 are wound in opposite senses. For example, the electrical conductor 18 may be wound with a right hand sense about the inner tubular member 14 with the winding 28 being wound in a left hand sense about the inner tubular member 14 or vice versa. With this arrangement, the flexibility of the electrical lead 10 is maintained while enhanced kink resistance of the electrical lead 10 is provided.

The winding 28 is of a biocompatible metal which is sufficiently dense to be radio opaque under X-rays. For example, the winding 28 is of tantalum, platinum, tungsten, or the like. The winding 28 is coated with insulation.

The sleeve 32 also provides a smoother, constant cross-section for the body member 12 of the electrical lead 10 and, as indicated above, serves to inhibit electrical cross-connection to the winding 28. The sleeve 32 is also of PEBAX®. The sleeve 32 may be of a softer grade of PEBAX® than that of the jacket 20 to maintain the flexibility of the electrical lead 10.

As indicated above, the electrical lead 10 functions as an electrode sheath 40 of a catheter 42. The catheter 42 includes a catheter handle 44 having a body 46. A first, steering control member 48 is mounted on an axially displaceable carrier 50 at a distal region of the body 46 with a second, projection control member 52 being carried distally of the steering control member 48 on the carrier 50. The steering control member 48 is fast with the carrier 50 while the projection control member 52 is displaceably arranged relative to the carrier 50. The electrode sheath 40 is carried on, and is fast with, a distal end of the projection control member 52.

A steering shaft (not shown) is received in the lumen 16 of the electrode sheath 40. The steering shaft is secured to the handle 44 via a mounting knob 54 and a slide (not shown) is displaceably arranged relative to the knob 54. The slide is fast with the carrier 50, and, hence, the steering control member 48, so that axial displacement of the steering control member 48 facilitates bending and steering of a distal end 54 of the electrode sheath 40. This facilitates steering of the electrode sheath 40 through the vascular system of a patient and also placement of the electrodes 22 at a site in the patient's body to be treated or for diagnostic purposes.

As described in applicant's co-pending Patent Application No. PCT/AU2006/000266 dated 1 Mar. 2006 and entitled “A catheter handle and a catheter assembly including such a handle”, it is beneficial to be able to extend the distal end 54 of the electrode sheath relative to the steering shaft contained in the lumen 16 of the electrode sheath 40. This has benefits, for example, to obtain improved tissue-electrode contact or to facilitate access to difficult to access sites in the patient's body. For this purpose, the projection control member 52 is provided. The distal end 54 is extended relative to the steering shaft by a distance of approximately 2 cm at most.

To provide improved kink resistance of the body member 11 of the electrode sheath 40 when extended relative to the steering shaft, the winding 28, proximally of a distal, tip electrode 22.1 (FIG. 3) of the lead 10, is coiled with a slightly narrower pitch than between other electrodes in the sequence of electrodes 22. Thus, more particularly, between a distal, tip electrode 22.1 and its adjacent electrode 22.2, the turns of the winding 28 are closer together than, for example, the turns of the winding 28 between electrodes 22.2 and 22.3. As indicated, this provides improved kink resistance when the distal end 54 of the electrode sheath 40 is in an extended position relative to the steering shaft under the action of the projection control member 52 of the catheter 42.

With the provision of the winding 28 of radio opaque material, when the electrode sheath 40 is inserted into the vasculature of the patient or is at the desired site to be treated, the markers 26 of the winding 28 provide improved radio opacity to enable a clinician to determine the position of the electrodes 22 associated with the relevant markers 26 in the patient's body.

Hence, it is an advantage of the invention that an electrical lead 10 is provided which has improved radio opaque characteristics. These improved radio opaque characteristics are provided in an electrical lead which is not significantly larger, in cross sectional diameter, than an electrical lead without such a radio opaque markers. Hence, the benefit of a more compact electrical lead 10 is still realised.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A medical use electrical lead which includes
an elongate body member comprising an inner tubular member defining a lumen and a cover member covering the inner tubular member;
at least one electrode defined on an outer surface of the cover member;
at least one pair of conductors arranged between the inner tubular member and the cover member, the at least one pair of conductors being in electrical communication with the at least one electrode through the cover member; and
a radio opaque marker circumscribing a periphery of the inner tubular member and underlying the at least one electrode.

2. The electrical lead of claim 1 in which the radio opaque marker is formed by an elongate element arranged about the tubular member, the elongate element being configured to provide radio-opacity at at least the position of the at least one electrode on the body member.

3. The electrical lead of claim 2 in which the elongate element is a winding of a radio opaque material.

4. The electrical lead of claim 3 in which turns of the winding have a smaller pitch in the region of the at least one electrode.

5. The electrical lead of claim 4 in which, at a distal region of the elongate body member, the winding has a pitch greater than the pitch of the winding in the region of the at least one electrode to provide kink resistance at the distal region of the elongate body member.

6. The electrical lead of claim 3 in which the elongate element is wound in an opposite sense to the at least one pair of conductors.

7. The electrical lead of claim 2 in which the elongate element underlies the at least one pair of conductors.

8. The electrical lead of claim 2 in which the elongate element is covered by a sleeve interposed between the inner tubular member and the cover member.

9. The electrical lead of claim 2 in which the elongate element carries an insulating layer.

10. A method of forming a medical use electrical lead, the method including
providing an inner tubular member;
arranging at least one pair of conductors helically about the tubular member;
covering the at least one pair of conductors with a cover member;
forming at least one electrode at least partially about the cover member at a predetermined position and placing the electrode in electrical communication with the at least one pair of conductors; and
arranging a radio opaque marker about a periphery of the tubular member to circumscribe a periphery of the tubular member beneath the at least one electrode to provide radio-opacity at the location of the at least one electrode.

11. The method of claim 10 which includes arranging the at least one radio opaque marker about the periphery of the tubular member prior to winding the at least one pair of conductors about the tubular member so that the at least one pair of conductors overlies the at least one radio opaque marker.

12. The method of claim 10 which includes forming the at least one radio opaque marker by winding an elongate element about the inner tubular member.

13. The method of claim 12 which includes winding the elongate element in an opposite sense to the at least one pair of conductors.

14. The method of claim 12 which includes, in the region of the at least one electrode, winding the elongate element with a smaller pitch.

15. The method of claim 14 which includes, at a distal region of the inner tubular member, winding the elongate element with a greater pitch than in the region of the at least one electrode to provide kink resistance to the distal region of the inner tubular member.

16. The method of claim 12 which includes interposing a sleeve between the inner tubular member and the cover member to at least partially cover the elongate element.

17. A catheter sheath which includes
an elongate body member comprising an inner tubular member defining a lumen and a cover member covering the inner tubular member;
at least one electrode defined on an outer surface of the cover member;
at least one pair of conductors arranged about the inner tubular member and covered by the cover member and in electrical communication with the at least one electrode through the cover member; and
a radio opaque marker circumscribing a periphery of the inner tubular member and underlying the at least one electrode.

18. A catheter which includes a catheter sheath as claimed in claim 17.

19. The catheter of claim 18 which is a cardiovascular catheter.
